(19) United States
(12) Patent Application Publication
(10) Pub. No.: US 2021/0005310 A1
(43) Pub. Date: Jan. 7, 2021

(54) ORDER CREATION SUPPORT APPARATUS AND ORDER CREATION SUPPORT METHOD

(71) Applicant: CANON MEDICAL SYSTEMS CORPORATION, Otawara-shi (JP)

(72) Inventors: Kazumasa NORO, Shioya-gun (JP); Kazuki UTSUNOMIYA, Nasushiobara (JP)

(73) Assignee: CANON MEDICAL SYSTEMS CORPORATION, Otawara-shi (JP)

(21) Appl. No.: 16/918,183

(22) Filed: Jul. 1, 2020

(30) Foreign Application Priority Data
Jul. 3, 2019 (JP) 2019-124553

(51) Int. Cl. G16H 40/20; G06Q 30/06; G06Q 10/06; G06Q 10/10; G16H 15/00

(52) U.S. Cl. CPC G16H 40/20; G06Q 30/0635; G06Q 10/0633; G16H 50/20; G06Q 10/06312; G16H 15/00; G06Q 10/10

(57) ABSTRACT

An order creation support apparatus according to an embodiment includes a processing circuitry. The processing circuitry extracts existing order information similar to input new order information, from pieces of the existing order information, based on the similarity between the new order information and the existing order information. The processing circuitry also extracts representation commonly used for a specific item, from a character string included in the extracted existing order information, as common representation, and displays the new order information and support information based on the extracted common representation.

### CROSS-REFERENCE TO RELATED APPLICATIONS

This application is based upon and claims the benefit of priority from Japanese Patent Application No. 2019-124553, filed on Jul. 3, 2019; the entire contents of which are incorporated herein by reference.

### FIELD

Embodiments described herein relate generally to an order creation support apparatus and an order creation support method.

### BACKGROUND

In general, in hospitals, when a patient is examined using a medical image diagnosis apparatus, a doctor creates order information and transmits the order information to a department in charge such as a radiology information system (RIS) where the examination takes place.

Moreover, conventionally, various technologies have been developed to prevent errors in the order information. For example, a technology has been developed to determine whether the input order information corresponds to error-prone order information, and issues a warning in such a case.

### BRIEF DESCRIPTION OF THE DRAWINGS

FIG. 1 is a diagram illustrating a configuration example of a medical information system according to an embodiment;

FIG. 2 is a diagram illustrating an example of items of order information according to the embodiment;

FIG. 3 is a diagram schematically illustrating an example of schedule information stored in a schedule information DB of the embodiment;

FIG. 4 is a diagram schematically illustrating an example of workflow information stored in a workflow information DB of the embodiment;

FIG. 5 is a diagram illustrating a configuration example of a hospital information apparatus according to the embodiment;

FIG. 6 is a diagram illustrating an example of a functional configuration of the hospital information apparatus according to the embodiment;

FIG. 7 is an explanatory diagram of an example of a vectorization process according to the embodiment;

FIG. 8 is an explanatory diagram of an example of a vectorization process according to the embodiment;

FIG. 9 is a diagram illustrating an example of calculation results of similarities by a similarity calculation function of the embodiment;

FIG. 10 is a diagram illustrating an example of workflow information extracted by a similarity information extraction function of the embodiment;

FIG. 11 is a diagram for explaining an operation of a common representation extraction function according to the embodiment;

FIG. 12 is a diagram for explaining an operation of the common representation extraction function according to the embodiment;

FIG. 13 is a diagram illustrating an example of schedule information extracted by the similarity information extraction function of the embodiment;

FIG. 14 is a diagram illustrating an example of a screen to be displayed by a display control function of the embodiment;

FIG. 15 is a flowchart illustrating an example of a process executed by the hospital information apparatus of the embodiment; and

FIG. 16 is a diagram illustrating an example of a screen to be displayed by a display control function of a first modification.

### DETAILED DESCRIPTION

An order creation support apparatus according to an embodiment includes a processing circuitry. The processing circuitry extracts existing order information similar to input new order information, from pieces of the existing order information, on the basis of the similarity between the new order information and the existing order information. The processing circuitry also extracts representation commonly used for a specific item, from a character string included in the extracted existing order information, as common representation, and displays the new order information and support information based on the extracted common representation.

Hereinafter, an embodiment of an order creation support apparatus and an order creation support method will be described in detail with reference to the accompanying drawings.

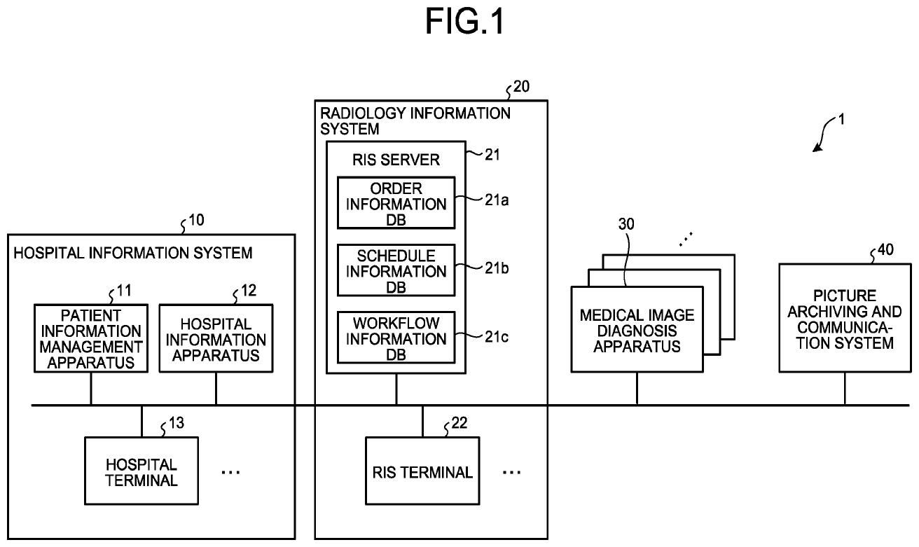

FIG. 1 is a diagram illustrating a configuration example of a medical information system according to the present embodiment. As illustrated in FIG. 1, a medical information system 1 includes a hospital information system (HIS) 10, a radiology information system (RIS) 20, a medical image diagnosis apparatus 30, a picture archiving and communication system (PACS) 40, and the like.

The hospital information system 10 is a system for managing the services, ordering, electronic health records, and the like of the entire hospital. For example, the hospital information system 10 includes a patient information management apparatus 11, a hospital information apparatus 12, and a hospital terminal 13.

The patient information management apparatus 11 manages information relating to patients (hereinafter, referred to as patient information). For example, the patient information records include items such as patient identifier (ID), patient name, gender, height, weight, age, and blood type. For example, the patient information management apparatus 11 is implemented by a computer apparatus such as a database apparatus and a server.

The hospital information apparatus 12 is an example of an order creation support apparatus. The hospital information apparatus 12 executes various information processes according to the order placed on the radiology information system 20. For example, the hospital information apparatus 12 is implemented by a computer apparatus such as a workstation, a server, and a personal computer.

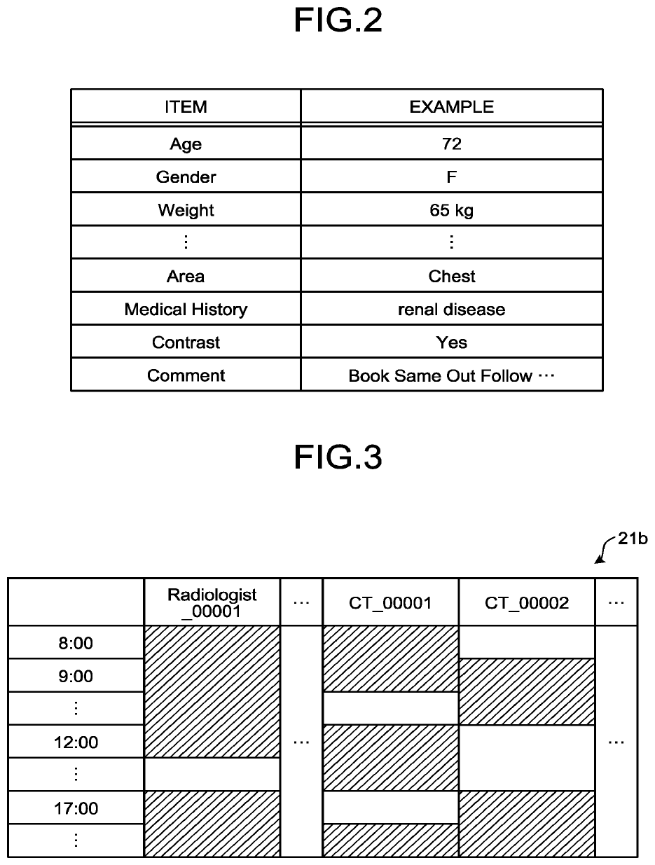

The hospital information apparatus 12 provides a user such as a clinical doctor with a screen used to support the creation of order information. Moreover, the hospital information apparatus 12 transmits the created order information to the radiology information system 20. In this example, the order information is used to request examination, and for example, as illustrated in FIG. 2, the order information includes items relating to a patient to be examined, items relating to the instruction of examination, and the like.

FIG. 2 is a diagram illustrating an example of items of order information. For example, the order information includes items relating to a patient such as the patient's age, gender, and weight. For example, these items are input on the basis of the patient's information in the patient information management apparatus 11. Moreover, the order information also includes various items such as an area to be examined, medical history of the patient, the presence of contrast, and a comment column. The contents of the order information are not limited to those illustrated in FIG. 2.

The hospital terminal 13 is connected to the patient information management apparatus 11 and the hospital information apparatus 12. For example, the hospital terminal 13 is used to carry out various services in the hospital information system 10 by a clinical doctor and the like. For example, the hospital terminal 13 includes an input interface, a display, and a communication interface.

The radiology information system 20 manages examination reservation information according to radiological examinations. The radiology information system 20 includes an RIS server 21 and an RIS terminal 22.

The RIS server 21 stores various types of information according to the radiological examinations in a storage in the server. More specifically, the storage of the RIS server 21 holds an order information database (DB) 21a, a schedule information DB 21b, and a workflow information DB 21c.

The order information DB 21a stores therein the order information transmitted from the hospital information system (hospital information apparatus 12). Upon receiving the order information from the hospital information system 10, the RIS server 21 issues an order number, and stores the received order information in association with the issued order number, in the order information DB 21a. In this example, the order number is a number issued when order information is input, and for example, is an identifier for uniquely specifying the order information in a single hospital.

The schedule information DB 21b stores therein the schedule information indicating the work schedule of a resource according to the radiological examinations such as the medical image diagnosis apparatus 30 and a radiologist. FIG. 3 is a diagram schematically illustrating an example of schedule information stored in the schedule information DB 21b. In FIG. 3, CT_00001 and CT_00002 are apparatus IDs for identifying each of a plurality of the medical image diagnosis apparatus 30. Moreover, Radiologist 00001 is an employee ID for identifying an employee such as a radiologist. For each medical image diagnosis apparatus 30 and an employee, the schedule information DB 21b stores therein the date and time when the examination is scheduled to take place, and the like, as schedule information. In FIG. 3, the hatched areas (time zones) are time zones when examinations are scheduled to take place.

In this example, the employee and the like may manually register the schedule. For example, the employee may register his/her schedule in the schedule information DB 21b via the RIS terminal 22. Moreover, the RIS server 21 may automatically register the schedule. In this case, for example, upon receiving new order information from the hospital information system 10, the RIS server 21 refers to the schedule information of each resource that can execute the examination instructed in the order information, and decides the resource in charge of the examination from the available schedule. Then, the RIS server 21 registers the time zone when the examination is scheduled to take place, in the schedule information of the resource in charge of the examination. Then, according to the schedule registered in the schedule information DB 21b, the resource carries out the examination as instructed in the order information.

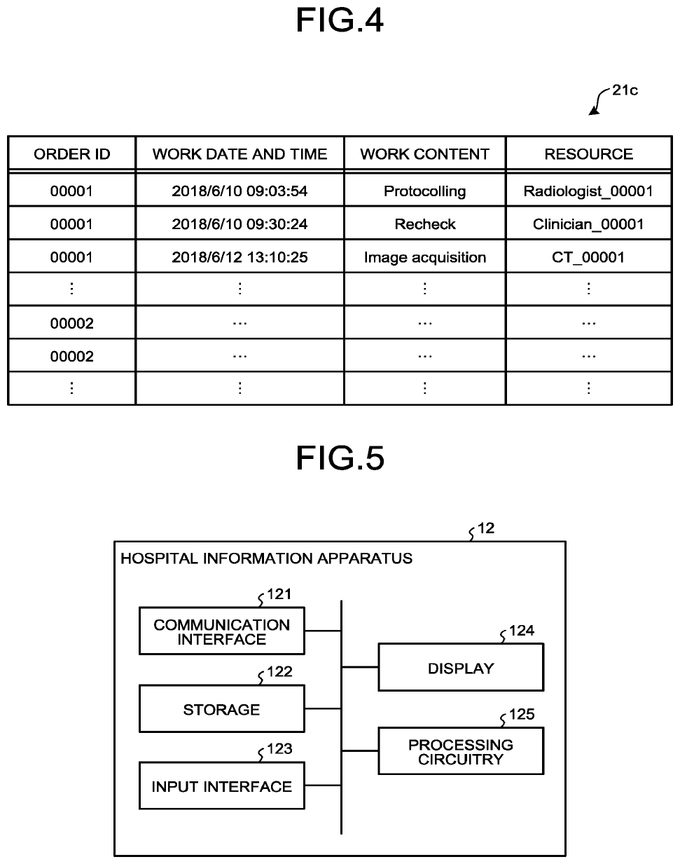

The workflow information DB 21c stores therein a series of work results performed on the basis of the order information, as workflow information. FIG. 4 is a diagram schematically illustrating an example of workflow information stored in the workflow information DB 21c. As illustrated in FIG. 4, the workflow information DB 21c stores therein workflow information including items such as order ID, work date and time, work content, and resource.

In the order ID, an order ID of the order information, which is to be carried out, is registered. In the work date and time and the work content, the date and time when the work was performed, and the work content are registered. Moreover, in the resource, a apparatus ID or an employee ID for specifying the resource that has carried out the work is registered. For example, in the example of FIG. 4, for the order information with the order ID “00001”, the fact that work for setting a protocol is performed by a radiologist with the employee ID “Radiologist 00001” on the work date and time “2018/6/10 09:03:54”, is recorded as the work result. Moreover, for the order information with the same order ID, the fact that a clinician with the employee ID “Clinician 00001” has rechecked the order information on the same day at “09:30:24”, is recorded as the work result. Furthermore, for the order information with the same order ID, the fact that image acquisition is performed by the medical image diagnosis apparatus 30 with the apparatus ID “CT 00001” on the work date and time “2018/6/12 13:10:25”, is recorded as the work result.

The employee and the like may manually register the work result via the RIS terminal 22 and the like, or the RIS server 21 may automatically register the work result. In case of the latter, for example, the RIS server 21 receives work result information transmitted from the resources, and registers the received work result information in the workflow information DB 21c in association with the reception date and time (work date and time). It is assumed that the work result information includes order ID, work content, apparatus ID, employee ID, or the like.

For example, the RIS terminal 22 is connected to the RIS server 21, and is used to carry out various services in the radiology information system 20 by a radiologist and the like. For example, the RIS terminal 22 includes an input interface, a display, and a communication interface.

Returning to FIG. 1, the medical image diagnosis apparatus 30 is a apparatus (modality) for carrying out an examination by taking an image of a patient and the like. For example, the medical image diagnosis apparatus 30 includes an X-ray computer tomographic apparatus, an X-ray diagnosis apparatus, a magnetic resonance imaging apparatus, a nuclear medical diagnosis apparatus, an ultrasound diagnosis apparatus, and the like. It is assumed that the dedicated apparatus ID described above is assigned to each medical image diagnosis apparatus 30 in advance.

For example, the medical image diagnosis apparatus 30 carries out an examination on the basis of the order information transmitted from the RIS server 21. Moreover, for example, the medical image diagnosis apparatus 30 generates work result information according to the operational state of the own apparatus, and transmits the work result information to the RIS server 21.

Furthermore, the medical image diagnosis apparatus 30 generates medical image data by carrying out an examination. For example, the medical image data includes X-ray computerized tomography (CT) image data, X-ray image data, magnetic resonance imaging (MRI) image data, nuclear medical image data, ultrasound image data, and the like. For example, the medical image diagnosis apparatus 30 generates a medical image file, by converting the generated medical image data into a format conforming to the digital imaging and communication in medicine (DICOM) standard. For example, the medical image file is in a format conforming to the DICOM standard. The medical image diagnosis apparatus 30 transmits the generated medical image file to the picture archiving and communication system 40.

The picture archiving and communication system 40 manages various medical image files. More specifically, the picture archiving and communication system 40 stores the medical image data transmitted from the medical image diagnosis apparatus 30 in a storage such as DB, which is not illustrated, and manages the medical image data.

Next, a configuration of the hospital information apparatus 12 described above will be explained. FIG. 5 is a diagram illustrating a configuration example of the hospital information apparatus 12. As illustrated in FIG. 5, the hospital information apparatus 12 includes a communication interface 121, a storage 122, an input interface 123, a display 124, and a processing circuitry 125.

The communication interface 121 is connected to the processing circuitry 125, and controls transmission and communication of various types of data performed between the hospital information apparatus 12 and an external apparatus. More specifically, the communication interface 121 controls transmission and communication of various types of data performed between the hospital information apparatus 12 and the apparatus in the hospital information system 10 such as the patient information management apparatus 11 and the hospital terminal 13. Moreover, the communication interface 121 controls transmission and communication of various types of data performed between the hospital information apparatus 12 and the RIS server 21 in the radiology information system 20. For example, the communication interface 121 is implemented by a network card, a network adapter, a network interface controller (NIC), and the like.

The storage 122 is connected to the processing circuitry 125, and stores therein various types of data and various programs. For example, the storage 122 is implemented by a semiconductor memory element such as a random access memory (RAM) and a flash memory, a hard disk, an optical disc, or the like. The storage 122 is an example of means for implementing a storage unit.

The input interface 123 is connected to the processing circuitry 125, and receives input operations of various instructions and various types of information from an operator. More specifically, the input interface 123 converts an input operation received from the operator to an electric signal, and outputs the electric signal to the processing circuitry 125. For example, the input interface 123 is implemented by a trackball, a switch button, a mouse, a keyboard, a touch pad that performs an input operation by touching an operation surface, a touch screen in which the display screen and the touch pad are integrated, a non-contact input circuit that uses an optical sensor, a voice input circuit, and the like. It is to be noted that the input interface 123 is not limited to those including a physical operation component such as a mouse and keyboard. Examples of the input interface 123 include an electric signal processing circuit that receives an electric signal corresponding to an input operation from an external input device provided separately from the device, and that outputs the electric signal to a control circuit.

The display 124 is connected to the processing circuitry 125, and displays various types of information and various images. More specifically, the display 124 converts data on various types of information and various images sent from the processing circuitry 125 into an electric signal for displaying, and outputs the electric signal. For example, the display 124 is implemented by a liquid crystal monitor, a cathode ray tube (CRT) monitor, a touch panel, and the like.

The processing circuitry 125 controls the components of the hospital information apparatus 12, according to the input operation received from the operator via the input interface 123. More specifically, the processing circuitry 125 causes the display 124 to display a screen used to create order information. For example, the processing circuitry 125 is implemented by a processor.

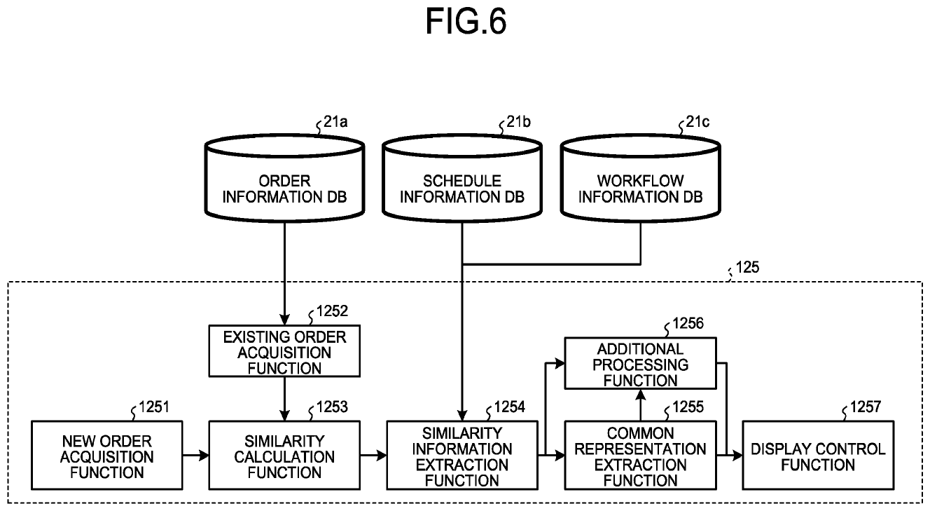

Moreover, the hospital information apparatus 12 has a function of supporting the user such as a clinical doctor to create order information, by using the order information DB 21a, the schedule information DB 21b, and the workflow information DB 21c managed by the RIS server 21. Hereinafter, with reference to FIG. 6, a functional configuration of the hospital information apparatus 12 will be described.

FIG. 6 is a diagram illustrating an example of a functional configuration of the hospital information apparatus 12. As illustrated in FIG. 6, the processing circuitry 125 of the hospital information apparatus 12 includes a new order acquisition function 1251, an existing order acquisition function 1252, a similarity calculation function 1253, a similarity information extraction function 1254, a common representation extraction function 1255, an additional processing function 1256, and a display control function 1257. The similarity calculation function 1253 is an example of a similarity calculation unit. The similarity information extraction function 1254 is an example of a first extraction unit. The common representation extraction function 1255 is an example of a second extraction unit. The additional processing function 1256 is an example of an operational state determination unit. The display control function 1257 is an example of a display control unit.

When order information is input anew (hereinafter, may also be referred to as new order information) via the input interface 123 and the like, the new order acquisition function 1251 acquires the new order information, and inputs the new order information to the similarity calculation function 1253. For example, upon receiving an operation instructing to check the input content after the new order information is input, the new order acquisition function 1251 acquires the input new order information. It is assumed that the new order acquisition function 1251 acquires the new order information, before the new order information is transmitted to the radiology information system 20.

The existing order acquisition function 1252 acquires the order information in existence (hereinafter, may also be referred to as existing order information), stored in the order information DB 21a of the RIS server 21, and inputs the existing order information to the similarity calculation function 1253. The number of pieces of existing order information acquired by the existing order acquisition function 1252 is not particularly limited, and for example, the existing order acquisition function 1252 may acquire the entire pieces of existing order information stored in the order information DB 21a. Alternatively, the existing order acquisition function 1252 may acquire the pieces of existing order information of the recent several years (for example, three years).

The similarity calculation function 1253 compares between the new order information acquired by the new order acquisition function 1251 and the existing order information acquired by the existing order acquisition function 1252, and calculates the similarity (or difference) between the two pieces of order information. In this example, the calculation method of the similarity is not particularly limited, and various methods may be used. In the following, an example will be described in which the similarity is calculated by vectorization of character strings used in the field of machine learning and the like.

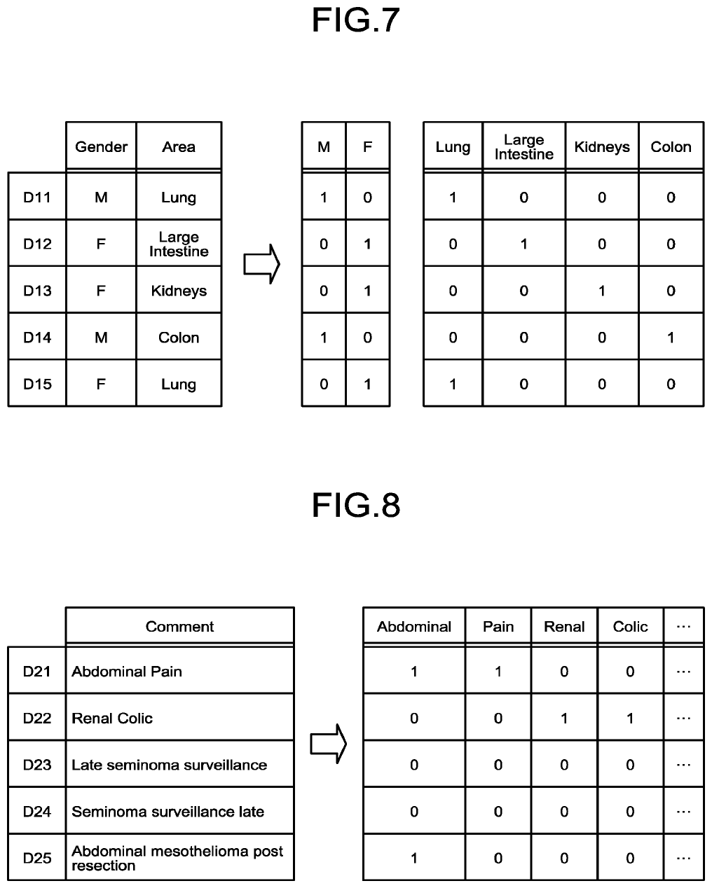

First, the similarity calculation function 1253 vectorizes the character strings included in the new order information and the existing order information by word or by context. The vectorization method is not particularly limited, and various methods may be used. Moreover, the vectorization method may be changed for each item included in the order information. For example, as illustrated in FIG. 7, each of the items such as gender and an area included in the order information may be vectorized by converting the item to one-hot representation.

FIG. 7 is an explanatory diagram of an example of a vectorization process, and schematically illustrates a vectorization method using the one-hot representation. In the one-hot representation, a numerical value is assigned to each word included in a predetermined category, and the character string in the same category corresponding to the word is converted to a corresponding numerical value. FIG. 7 illustrates an example in which, for each word of “M” and “F” included in the category of gender, 1 is assigned if applicable and 0 is assigned otherwise. Moreover, FIG. 7 also illustrates an example in which, for each word of “lung”, “large intestine”, “kidneys”, and “colon” included in the category of area, 1 is assigned if applicable and 0 is assigned otherwise.

For example, in the order information D11 to D15 illustrated in the left of FIG. 7, because the gender of the order information D11 is “M”, on the basis of a conversion rule of the gender category, the order information is vectorized to [1, 0]. Moreover, because the examination area of the order information D11 is “lung”, on the basis of a conversion rule of the examination area category, the order information is vectorized to [1, 0, 0, 0]. Furthermore, for the order information D12, because the gender is “F” and the examination area is “large intestine”, on the basis of conversion rules of the gender category and the examination area category, the order information is vectorized to [0, 1] and [0, 1, 0, 0]. The order information D13 to D15 can also be vectorized by category as described above.

Moreover, another vectorization method may also be used for an item where any character string such as a comment can be inserted, because the item may include a sentence including a plurality of words and a character string including words other than fixed words. For example, vectorization may be performed using a bag-of-words method.

FIG. 8 is an explanatory diagram of an example of a vectorization process, and schematically illustrates a vectorization method using the bag-of-words method. In the bag-of-words method, a numerical value is assigned to each word obtained by analyzing a character string described in natural language using morpheme analysis and the like. FIG. 8 illustrates an example in which, for each of the words in character strings of “abdominal”, “pain”, “renal”, “colic”, . . . , 1 is assigned if applicable and 0 is assigned otherwise. For example, in the order information D21 to D25 illustrated in the left of FIG. 8, because the comment content of the order information D21 is “abdominal pain”, on the basis of the conversion rule described above, the order information is vectorized to [1, 1, 0, 0, . . . ]. Moreover, because the comment content of the order information D22 is “renal colic”, on the basis of the conversion rule described above, the order information is vectorized to [0, 0, 1, 1, . . . ]. The order information D23 to D25 can also be vectorized as described above.

When the new order information and the existing order information are vectorized, the similarity calculation function 1253 calculates the similarity between the new order information and the existing order information, by comparing between the vectorized new order information and the vectorized existing order information. For example, the similarity calculation function 1253 may calculate the similarity between the new order information and the existing order information, by using cosine similarity. Moreover, for example, the similarity calculation function 1253 may calculate the similarity between the new order information and the existing order information, by using a technique such as Word2Vec or Doc2Vec that is used in the fields of neural networks and machine learning. Furthermore, for example, the similarity calculation function 1253 may also calculate the similarity between the new order information and the existing order information, by using Euclidean distance.

When the order information is vectorized by item, the similarity calculation function 1253 may calculate the similarity between the pieces of order information, by comparing between the same items, calculating the similarity for each item, and integrating the calculated similarities.

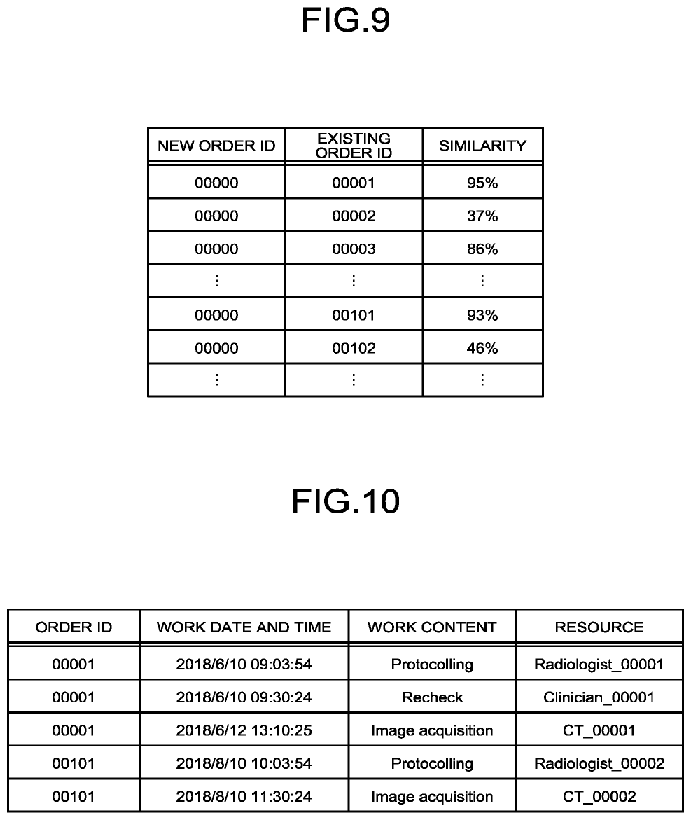

FIG. 9 is a diagram illustrating an example of calculation results of similarities by the similarity calculation function 1253. In FIG. 9, a new order ID is the order ID of new order information, and an existing order ID is the order ID of existing order information. In the present embodiment, the order ID is issued by the radiology information system 20. Consequently, at the step of the hospital information apparatus 12, the order ID of the new order information is not issued yet. Thus, FIG. 9 illustrates an example in which a temporary order ID (00000) is applied to the new order information for convenience sake.

As described above, the similarity calculation function 1253 calculates the similarity between two pieces of order information, by comparing between the new order information and the existing order information. For example, FIG. 9 illustrates calculation results of similarities between the new order information with the order ID “00000” and the pieces of existing order information with the order IDs of “00001”, “00002”, “00003” “00101”, “00102” In this example, the similarity is an index indicating the intensity of correspondence and correlation between the new order information and the existing order information. The similarity is increased with an increase in the number of common descriptions and similar descriptions.

Returning to FIG. 6, the similarity information extraction function 1254 extracts the existing order information similar to the new order information, and the workflow information and schedule information relating to the existing order information, on the basis of the similarity calculated by the similarity calculation function 1253.

More specifically, the similarity information extraction function 1254 extracts the existing order information with the order ID the similarity of which is a threshold or more, from the pieces of existing order information (or order information DB 21a) acquired by the existing order acquisition function 1252, on the basis of the calculation result of the similarity calculation function 1253. For example, if the threshold is 90%, the similarity information extraction function 1254 extracts the pieces of existing order information with the order IDs “00001” and “00101” the similarity of which is the threshold or more, from the order IDs of the pieces of existing order information illustrated in FIG. 9.

Moreover, the similarity information extraction function 1254 extracts the workflow information including the order ID of the extracted existing order information, from the workflow information DB 21c. For example, when the pieces of existing order information with the order IDs “00001” and “00101” are extracted as described above, the similarity information extraction function 1254 extracts the pieces of workflow information including the order ID “00001” and the order ID “00101” from the schedule information DB 21b.

FIG. 10 is a diagram illustrating an example of workflow information extracted by the similarity information extraction function 1254. FIG. 10 illustrates an example in which the pieces of workflow information including the order IDs “00001” and “00101” are extracted. The pieces of workflow information extracted in this example indicate a series of work results performed in response to the pieces of existing order information with the order IDs “00001” and “00101”.

Moreover, the similarity information extraction function 1254 extracts the schedule information of the corresponding resource, on the basis of the resource ID included in the extracted workflow information, from the schedule information DB 21b. For example, if the extraction results of the workflow information are as illustrated in FIG. 10, the similarity information extraction function 1254 extracts the pieces of schedule information corresponding to the IDs of “Radiologist 00001”, “Clinician 00001”, and “CT 00001” included in the workflow information with the order ID “00001”, from the schedule information DB 21b. Furthermore, the similarity information extraction function 1254 extracts the pieces of schedule information corresponding to the IDs of “Radiologist 00002” and “CT 00002” included in the workflow information with the order ID “00101”, from the schedule information DB 21b. As for the resource not registered in the schedule information DB 21b such as “Clinician 00001”, it is not necessary to extract the schedule information.

Returning to FIG. 6, the common representation extraction function 1255 extracts the representation commonly used for an item that is specific (hereinafter, a specific item) from the character string included in the order information extracted by the similarity information extraction function 1254, as the common representation. In this example, the specific item is not particularly limited. For example, the specific item can be set optionally from the items in the order information extracted by the similarity information extraction function 1254, the other information (such as the workflow information) relating to the order information, and the like.

Moreover, an extraction method of the common representation is not particularly limited, and various methods may be used. For example, when a character string used for a specific item is expressed by a numerical value such as dose, the common representation extraction function 1255 may extract the average value of the corresponding numerical values included in the pieces of existing order information similar to the new order information, as the common representation. Moreover, for example, the common representation extraction function 1255 may first divide the pieces of order information into a plurality of groups on the basis of the presence of the specific item or the setting content, and then extract the common representation for each divided group. Hereinafter, the latter extraction method will be described.

For example, among the items included in the workflow information, if the specific item is “recheck” indicating to recheck the order information, the common representation extraction function 1255 executes the following process.

First, the common representation extraction function 1255 divides the pieces of order information extracted by the similarity information extraction function 1254 into two groups, according to whether “recheck” is included in the work content of the workflow information. For example, it is assumed that the pieces of workflow information extracted by the similarity information extraction function 1254 are in the state as illustrated in FIG. 10. In this case, the work content “recheck” is present in the workflow information with the order ID “00001”, and the work content “recheck” is not present in the workflow information with the order ID “00101”. Consequently, the common representation extraction function 1255 divides the pieces of order information extracted by the similarity information extraction function 1254 into a group A with the order ID “00001” and a group B with the order ID “00101”.

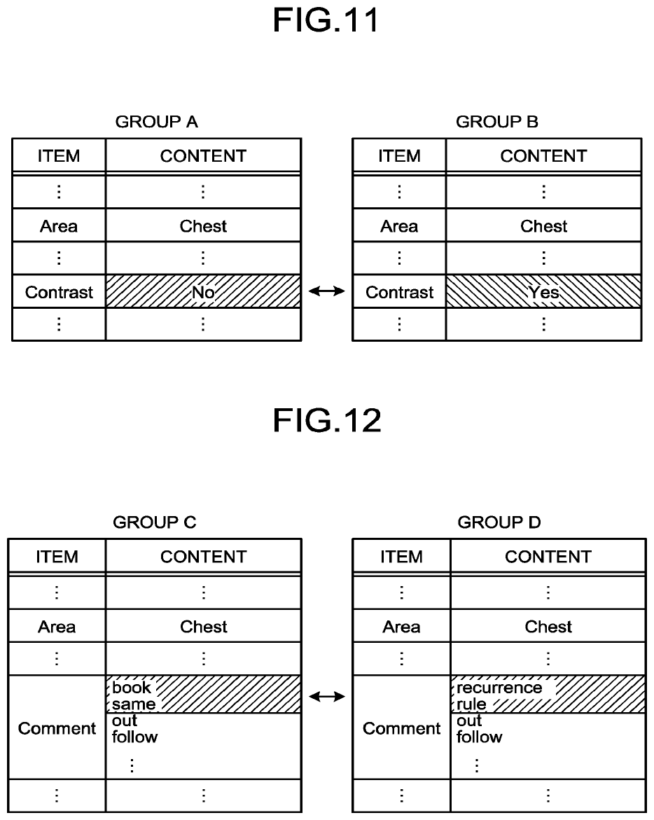

Next, the common representation extraction function 1255 compares the content of the order information extracted by the similarity information extraction function 1254 between the groups A and B, and extracts the content different between the groups A and B, as the common representation. For example, it is assumed that the order information (order ID “00001”) of the group A is in the state as illustrated in the left of FIG. 11, and the order information (order ID “00101”) of the group B is in the state as illustrated in the right of FIG. 11. In this example, FIG. 11 is a diagram for explaining an operation of the common representation extraction function 1255.

In FIG. 11, the common representation extraction function 1255 determines that the setting content relating to the item “contrast” differs between the group A and the group B, and extracts the setting content as the common representation. More specifically, the common representation extraction function 1255 extracts the setting content “No” in the item “contrast” included in the order information of the group A as the common representation of the order information to be rechecked. Moreover, the common representation extraction function 1255 extracts the setting content “Yes” of the item “contrast” included in the order information of the group B as the common representation of the order information not to be rechecked. In this manner, by extracting the common representation from the past order information that has been rechecked, it is possible to extract the representation in the order information that has caused the recheck.

Moreover, as another example, when the apparatus ID written in the resource is set as the specific item, among the items included in the workflow information, the common representation extraction function 1255 executes the following process.

First, according to the setting content (for example, a type of the apparatus ID) of the resource included in the workflow information, the common representation extraction function 1255 divides the pieces of order information extracted by the similarity information extraction function 1254 into a plurality of groups. For example, it is assumed that the workflow information extracted by the similarity information extraction function 1254 is in the state as illustrated in FIG. 10. In this case, the apparatus ID “CT 00001” is included in the workflow information with the order ID “00001”, and the apparatus ID “CT 00002” is included in the workflow information with the order ID “00101”. Consequently, the common representation extraction function 1255 divides the pieces of order information into a group C with the order ID “00001” and a group D with the order ID “00101”.

Next, the common representation extraction function 1255 compares the content of the order information extracted by the similarity information extraction function 1254 between the groups C and D, and extracts the content different between the groups C and D, as the common representation. For example, it is assumed that the order information (order ID “00001”) of the group C is in the state as illustrated in the left of FIG. 12, and the order information (order ID “00101”) of the group D is in the state as illustrated in the right of FIG. 12. In this example, FIG. 12 is a diagram for explaining an operation of the common representation extraction function 1255.

In case of FIG. 12, the common representation extraction function 1255 determines that a part of the comment content of the item “comment” is different, and extracts the setting content as the common representation. More specifically, the common representation extraction function 1255 determines that the words “book, same” included in the comment of the group C are different from the words “recurrence, rule” included in the comment of the group D. In this case, the common representation extraction function 1255 extracts the words “book, same” of the group C as the common representations used when an examination is carried out by the medical image diagnosis apparatus 30 with the apparatus ID “CT 00001”. Moreover, the common representation extraction function 1255 extracts the words “recurrence, rule” of the group D as the common representations used when an examination is carried out by the medical image diagnosis apparatus 30 with the apparatus ID “CT 00002”. In this manner, by extracting the common representation from the past order information in which the same medical image diagnosis apparatus 30 was used, it is possible to extract the representation (word) according to the selection of the medical image diagnosis apparatus 30.

In the example described above, the common representation is extracted on the basis of the apparatus ID.

However, it is not limited thereto, and the common representation may also be extracted on the basis of the employee ID. Moreover, when the pieces of order information are included in a divided group, the common representation extraction function 1255 may standardize the representation included in the order information in the same group, and compare between the groups. Furthermore, when the order information can only be divided into a single group, or when the existing order information is not extracted by the similarity information extraction function 1254, the common representation extraction function 1255 outputs “null” indicating that there is no common representation.

Returning to FIG. 6, the additional processing function 1256 executes an additional process to support the creation of new order information, in addition to the process of the common representation extraction function 1255. More specifically, when the common representation relating to the resource is extracted by the common representation extraction function 1255, the additional processing function 1256 determines the operational state of each resource from which the common representation is to be extracted, on the basis of the schedule information extracted by the similarity information extraction function 1254.

For example, it is assumed that the common representation extraction function 1255 has extracted the common representation of the group C and the group D (see FIG. 12). In this case, the additional processing function 1256 determines the operational state of the corresponding resource, on the basis of the schedule information of the apparatus IDs “CT_00001” and “CT_00002” of the groups C and D.

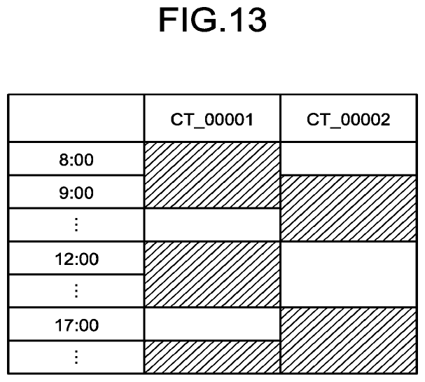

For example, it is assumed that the pieces of schedule information of the apparatus IDs “CT_00001” and “CT_00002” extracted by the similarity information extraction function 1254 are in the state as illustrated in FIG. 13. FIG. 13 is a diagram illustrating an example of schedule information extracted by the similarity information extraction function 1254.

In this example, the additional processing function 1256 determines the operational state of the corresponding resource, on the basis of the pieces of schedule information of the apparatus IDs “CT_00001” and “CT_00002”. For example, from the schedule within a predetermined period from the current date and time, the additional processing function 1256 determines the operational state of the resource on the basis of the ratio of the estimated work (examination) time.

For example, it is assumed that the ratio of the estimated work time of the apparatus ID “CT_00001” is 75%, the ratio of the estimated work time of the apparatus ID “CT_00002” is 50%, and a threshold for determining whether the apparatus is busy is 70%. In this case, the additional processing function 1256 determines that the apparatus ID “CT_00001” is busy, and the apparatus ID “CT_00002” is available.

It is to be noted that the process executed by the additional processing function 1256 is not limited to the example described above, and the additional processing function 1256 may also execute various processes according to the processing content of the common representation extraction function 1255.

Returning to FIG. 6, the display control function 1257 causes the display 124 to display various screens used to support the creation of new order information, on the basis of the processing results of the common representation extraction function 1255 and the additional processing function 1256.

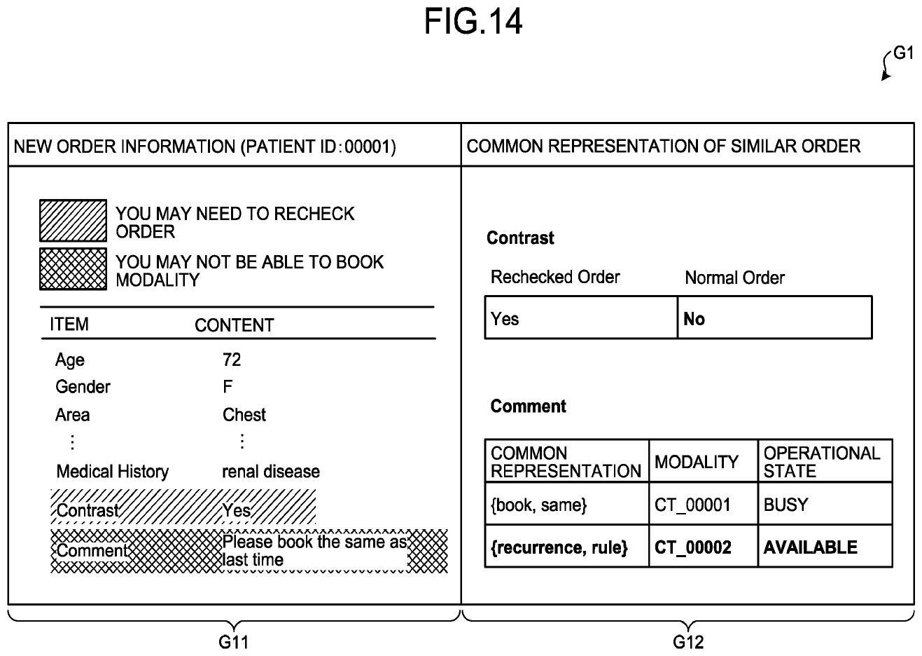

FIG. 14 is a diagram illustrating an example of a screen to be displayed by the display control function 1257. As illustrated in FIG. 14, a screen G1 includes an order content display area G11 and a support information display area G12.

Items included in the order information are displayed on the order content display area G11. The order content display area G11 is used for inputting a character string in each item, editing the input character string, and the like, via the input interface 123 and the like. The user of the hospital information apparatus 12 can create new order information via the order content display area G11.

Alternatively, support information used to support the input of order information, on the basis of the processing results of the common representation extraction function 1255 and the additional processing function 1256, is displayed on the support information display area G12.

For example, it is assumed that for the work content “recheck”, the common representation of the item “contrast” included in the order information is extracted by the common representation extraction function 1255. In this case, the display control function 1257 displays the support information relating to the item “contrast” on the support information display area G12, on the basis of the common representation extracted by the common representation extraction function 1255. In this example, as the support information, the support information display area G12 displays that the order information is rechecked when the setting content is “Yes”, and the order information is not rechecked when the setting content is “No”. Moreover, the display control function 1257 highlights and displays the item “contrast” in the order content display area G11 corresponding to the item “contrast” in the support information display area G12 as an item that needs to be checked again.

In this manner, the user of the hospital information apparatus 12 can easily correct the representation (setting) that causes the recheck, by confirming the input content in the order content display area G11, on the basis of the support information according to the item “contrast” displayed on the support information display area G12.

Moreover, for example, it is assumed that for the setting content of the resource (a type of the apparatus ID), the common representation of the item “comment” included in the order information is extracted by the common representation extraction function 1255, and the operational state of each resource is determined by the additional processing function 1256. In this case, on the basis of the processing results of the common representation extraction function 1255 and the additional processing function 1256, the display control function 1257 displays the support information relating to the resources (modalities) “CT_00001” and “CT_00002” on the support information display area G12. In this example, as the support information, the representation (word) of the comment input when “CT_00001” and “CT_00002” are used, and the operational states of CT_00001 and CT_00002 are displayed in an associated manner. Moreover, the display control function 1257 highlights and displays the comment (character string) on the order content display area G11 corresponding to the common representation “book, same” as the representation indicating that the apparatus may not be available.

In this manner, on the basis of the support information according to the medical image diagnosis apparatus 30 displayed on the support information display area G12, the user of the hospital information apparatus 12 can change the content to the representation (word) with which the available medical image diagnosis apparatus 30 can be selected, by confirming the comment content on the order content display area G11. Moreover, for example, if it is possible to specify the medical image diagnosis apparatus 30 via order information, the user of the hospital information apparatus 12 can specify the available medical image diagnosis apparatus 30 on the basis of the support information described above. Consequently, it is possible to prevent a situation such that bookings and processes are concentrated on a specific medical image diagnosis apparatus 30.

For example, after editing the content of the new order information on the basis of the support information, the user of the hospital information apparatus 12 instructs to complete the creation of the new order information via the input interface 123 and the like. Upon receiving the instruction to complete the creation, the processing circuitry 125 of the hospital information apparatus 12 transmits the content input to the order content display area G11, to the radiology information system 20 as the new order information.

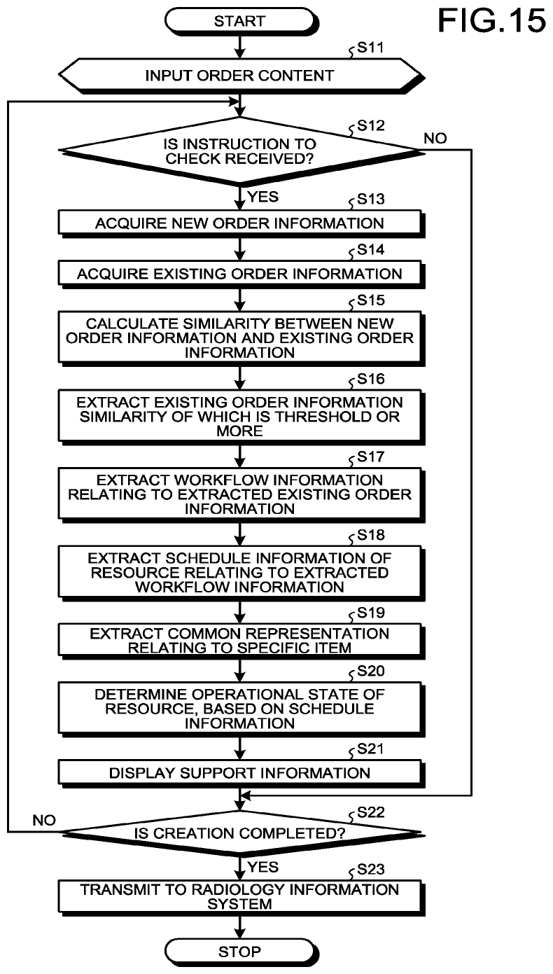

Hereinafter, with reference to FIG. 15, an example of a process executed by the hospital information apparatus 12 will be described. In this example, FIG. 15 is a flowchart illustrating an example of a process executed by the hospital information apparatus 12. As a prerequisite of the present process, it is assumed that order content is input to the order content display area G11 on the screen G1 (step S11).

First, the new order acquisition function 1251 determines whether an instruction to check the input content is received, via the input interface 123 and the like (step S12). In this process, if the instruction to check the input content is not received, the process proceeds to step S22.

If the instruction to check the input content is received (Yes at step S12), the new order acquisition function 1251 acquires the order content input to the order content display area G11 as the new order information (step S13). Moreover, according to the process of the new order acquisition function 1251, the existing order acquisition function 1252 acquires the existing order information from the order information DB 21a (step S14).

Next, the similarity calculation function 1253 calculates the similarity between the new order information acquired at step S13, and the existing order information acquired at step S14 (step S15). Next, on the basis of the similarity calculated at step S15, the similarity information extraction function 1254 extracts the existing order information the similarity of which is a threshold or more (step S16). Moreover, the similarity information extraction function 1254 extracts the workflow information including the order ID of the existing order information extracted at step S16, from the workflow information DB 21c (step S17). Furthermore, the similarity information extraction function 1254 extracts the schedule information of the resource included in the workflow information extracted at step S17, from the schedule information DB 21b (step S18).

Next, the common representation extraction function 1255 extracts the common representation relating to a specific item, from the existing order information extracted at step S17 (step S19). Moreover, when the common representation relating to the resource is extracted at step S19, the additional processing function 1256 determines the operational state of the resource, on the basis of the schedule information of the corresponding resource extracted at step S18 (step S20).

Next, the display control function 1257 displays the support information generated on the basis of the processing results at step S19 and step S20, on the support information display area G12 (step S21). On the basis of the support information displayed on the support information display area G12, the user of the hospital information apparatus 12 can edit the content of the new order information displayed on the order content display area G11.

Next, the processing circuitry 125 of the hospital information apparatus 12 determines whether the instruction to complete the creation of the new order information is received, via the input interface 123 and the like (step S22). In this process, when the instruction to complete the creation of the new order information is not received (No at step S22), the process is returned to step S12. On the other hand, when the instruction to complete the creation of the new order information is received (Yes at step S22), the processing circuitry 125 transmits the content input to the order content display area G11, to the radiology information system 20 as the new order information (step S23), and finishes the present process.

As described above, in the present embodiment, the existing order information similar to the new order information is extracted, the common representation used for the specific item is extracted from the character string included in the extracted existing order information, and the support information generated on the basis of the common representation is displayed with the new order information. Consequently, in the present embodiment, the common representation according to the content of the new order information can be extracted from the existing order information, every time new order information is created. Thus, it is possible to support the creation of order information from various viewpoints. As a result, with the present embodiment, it is possible to flexibly support the creation of order information.

The embodiment described above may also be suitably modified and executed, by changing a part of the configuration or function of the hospital information apparatus 12. Thus, in the following description, some modifications according to the embodiment described above will be explained. In the following description, points different from the embodiment described above are mainly explained, and the detailed descriptions of the same contents as those described above will be omitted. Moreover, the modifications described below may be carried out individually, or may be carried out by being combined with one another as appropriate.

### First Modification

In the embodiment described above, the creation of the order information to be transmitted to the radiology information system 20 is supported. However, it is not limited thereto, and the embodiment is also applicable to create the order information formed by the other system or the order information for other usage.

For example, the embodiment is applicable to support the creation of the order information for the medical image diagnosis apparatus 30 (radiologist), created by a radiology specialist of the radiology information system 20. In this case, for example, it is possible to support the creation of order information, when the apparatus such as the RIS server 21 and the medical image diagnosis apparatus 30 has a functional configuration similar to that of the hospital information apparatus 12 described above.

More specifically, the new order acquisition function 1251 acquires the order information created by a radiology specialist. Such order information is order information for requesting a treatment, and for example, includes the items relating to the patient such as “age”, “gender”, and “weight”. Moreover, for example, the order information includes items such as “protocol” according to the examination. Furthermore, for example, the order information includes the items relating to the photographing conditions such as “dose” and “contrast” used for the examination (photographing). Still furthermore, for example, the order information may also include the items for specifying the apparatus ID of the medical image diagnosis apparatus 30 used for the examination. It is assumed that a unique order ID is assigned to each piece of the order information.

The existing order acquisition function 1252 acquires the existing order information from the storage such as the DB that stores therein the existing order information. Moreover, the similarity calculation function 1253 calculates the similarity between the new order information acquired by the new order acquisition function 1251 and the existing order information acquired by the existing order acquisition function 1252. The same method as that in the embodiment described above may be used for the calculation method of the similarity.

The similarity information extraction function 1254 extracts the existing order information the similarity of which is a threshold or more, among the similarities calculated by the similarity calculation function 1253. Moreover, the similarity information extraction function 1254 extracts the workflow information corresponding to the order ID of the extracted order information, from the workflow information DB 21c and the like.

The common representation extraction function 1255 extracts the representation commonly used for the specific item, from the character string included in the extracted existing order information, as the common representation. It is to be noted that the specific item is not particularly limited, and may be set optionally. For example, it is also possible to set a specific protocol included in the order information as the specific item, and extract the average value of dose included in the existing order information, as the common representation relating to the specific item. Moreover, the same method as that of the embodiment described above may be used for the extraction method of the common representation.

The additional processing function 1256 executes various additional processes, in addition to the process of the common representation extraction function 1255. For example, when the average value of dose is extracted by the common representation extraction function 1255 as the common representation, the additional processing function 1256 may read out the content included in the pieces of existing order information used for calculating the average value.

The display control function 1257 causes the display of the RIS server 21, the RIS terminal 22, or the like to display various screens used to support the creation of order information, on the basis of the processing results of the common representation extraction function 1255 and the additional processing function 1256.

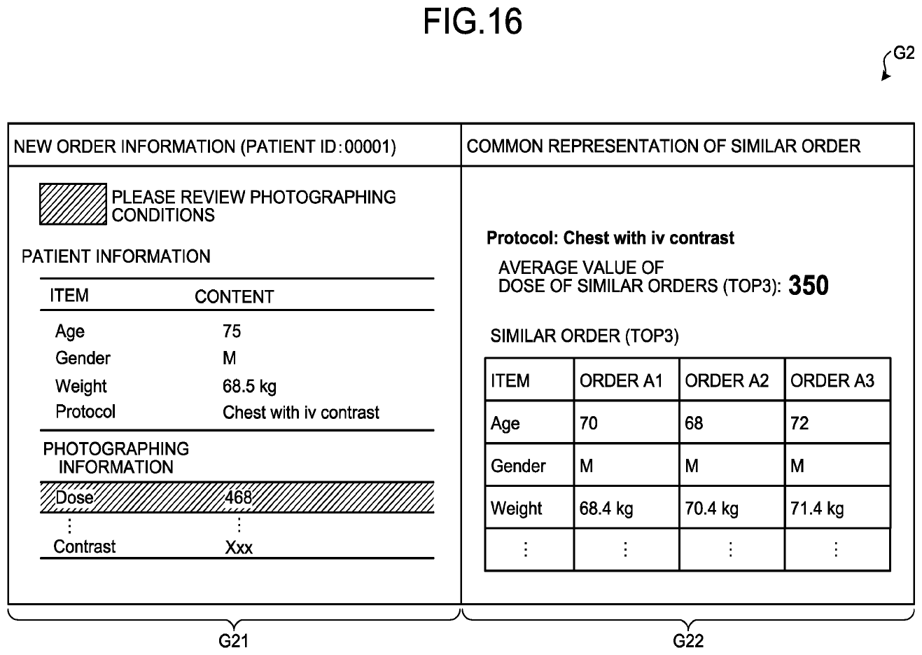

FIG. 16 is a diagram illustrating an example of a screen to be displayed by the display control function 1257 of the present modification. As illustrated in FIG. 16, a screen G2 includes an order content display area G21 and a support information display area G22.

Items included in the order information are displayed on the order content display area G21. The order content display area G21 is used for inputting a character string in each item, editing the input character string, and the like, via the input interface and the like. The user (radiology specialist) of the RIS server 21 or the RIS terminal 22 can create new order information via the order content display area G21.

Alternatively, on the support information display area G22, the support information used to support the input of order information is displayed, on the basis of the processing results of the common representation extraction function 1255 and the additional processing function 1256.

For example, it is assumed that the protocol “chest with iv contrast” included in the order information is set as the specific item, and the common representation relating to the dose is extracted by the common representation extraction function 1255. In this case, for example, the display control function 1257 displays the support information as illustrated in FIG. 16 on the support information display area G22, on the basis of the extraction result of the common representation extraction function 1255.

In this example, FIG. 16 illustrates a display example in which the average value of dose included in the pieces of order information with the top three similarities, among the pieces of existing order information including the protocol “chest with iv contrast” is extracted as the common representation. More specifically, the display control function 1257 displays the average value “350” of the dose extracted by the common representation extraction function 1255 on the support information display area G22, as the support information. Moreover, the display control function 1257 highlights and displays the item “dose” on the order content display area G21 corresponding to the dose of the support information display area G22, as an item the setting content of which can be reviewed.

In this manner, the user who creates the order information can easily correct the representation (setting) that causes excessive dose or the like, by changing the input content in the order content display area G21, on the basis of the value (average value) of dose displayed on the support information display area G22.

Moreover, the display control function 1257 displays the content of the top three pieces of order information (orders A1, A2, and A3) read out by the additional processing function 1256, as the support information. Consequently, the user who creates the order information can easily confirm and review the setting content according to the examination of dose or the like, by comparing between the information of the patient displayed on the support information display area G22, and the information of the patient input to the order content display area G21.

In the present modification, the additional processing function 1256 reads out the existing order information according to the calculated average value of dose, as the additional process. However, the additional process of the additional processing function 1256 is not limited thereto. For example, similar to the embodiment described above, the additional processing function 1256 may execute a process of determining the operational state of the resource such as the medical image diagnosis apparatus 30 and the radiologist.

In this manner, the user who creates the order information can specify the available medical image diagnosis apparatus 30 on the basis of the operational state of the resource displayed on the support information display area G22 and the like. Consequently, it is possible to prevent a situation such that bookings are concentrated on a specific medical image diagnosis apparatus 30.

### Second Modification

In the embodiment described above, the hospital information apparatus 12 includes the new order acquisition function 1251, the existing order acquisition function 1252, the similarity calculation function 1253, the similarity information extraction function 1254, the common representation extraction function 1255, the additional processing function 1256, and the display control function 1257. However, the order creation support apparatus other than the hospital information apparatus 12 may also include these functions.

For example, the hospital terminal 13 may function as the order creation support apparatus, by including the functions described above. Moreover, for example, the hospital information apparatus 12, which is the order creation support apparatus, may cause the display of the hospital terminal 13 to display the various screens described above, in the form of a Web service and the like. Furthermore, the order creation support apparatus is not limited to a single computer apparatus, and may be implemented by a plurality of computer apparatus connected to a network. In this case, each of the computer apparatus may include a part of the functions described above, and implement the order creation support apparatus as a whole.

In the embodiments described above, the similarity calculation unit, the first extraction unit, the second extraction unit, the operational state determination unit, and the display control unit of the present specification are respectively implemented by the similarity calculation function 1253, the similarity information extraction function 1254, the common representation extraction function 1255, the additional processing function 1256, and the display control function 1257 of the processing circuitry 125. However, the embodiment is not limited thereto. For example, the functions of the similarity calculation unit, the first extraction unit, the second extraction unit, the operational state determination unit, and the display control unit of the present specification may also be implemented by hardware only or by a combination of hardware and software, in addition to being implemented by the similarity calculation function 1253, the similarity information extraction function 1254, the common representation extraction function 1255, the additional processing function 1256, and the display control function 1257 described in the embodiment.

Moreover, for example, the word “processor” used in the explanation described above refers to a circuit such as a central processing unit (CPU), a graphics processing unit (GPU), an application specific integrated circuit (ASIC), or a programmable logic device (for example, a simple programmable logic device (SPLD), a complex programmable logic device (CPLD), and a field programmable gate array (FPGA)). The processor implements functions by reading out computer programs stored in the storage 122, and executing the computer programs. The computer programs may also be incorporated in the circuit of the processor directly, instead of storing the computer programs in the storage 122. In this case, the processor implements functions by reading out the computer programs incorporated in the circuit, and executing the computer programs. Moreover, the processor in the present embodiment is not always configured as a single circuit, but may also be configured as a single processor by combining a plurality of independent circuits, and implementing the functions.

In this example, the computer programs executed by the processor is provided by being incorporated in a read only memory (ROM), a storage, and the like in advance. The computer programs may also be provided by being recorded in a computer readable storage medium such as a compact disc (CD)-ROM, a flexible disk (FD), a CD-recordable (CD-R), and a digital versatile disc (DVD), in a file format installable or executable in these devices. Moreover, the computer programs may be provided or distributed by being stored on a computer connected to a network such as the Internet, and by being downloaded through the network. For example, each of the computer programs has a module configuration including the functional units described above. As actual hardware, the CPU reads out the computer program from the storage medium such as ROM, and executes the computer program so that each module is loaded on the main storage, and is generated on the main storage.

With at least one of the embodiment and modifications described above, it is possible to more flexibly support the creation of order information.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An order creation support apparatus, comprising:
a processing circuitry configured to
extract existing order information similar to input new order information, from pieces of the order information, based on similarity between the new order information and the existing order information;
extract representation commonly used for a specific item, from a character string included in the extracted existing order information, as common representation; and
display the new order information and support information based on the extracted common representation.

2. The order creation support apparatus according to claim 1, wherein the processing circuitry
extracts workflow information performed in response to the existing order information similar to the new order information, from pieces of the workflow information in which a work result performed in response to the existing order information is recorded, and
extracts the common representation relating to the specific item included in the extracted workflow information.

3. The order creation support apparatus according to claim 2, wherein the processing circuitry
divides the extracted workflow information into a plurality of groups, according to presence of the specific item or a setting content, and
extracts the common representation relating to the specific item, from each of the divided groups.

4. The order creation support apparatus according to claim 3, wherein the processing circuitry
divides the existing order information corresponding to the workflow information into two groups, according to whether the workflow information includes a work result instructing to recheck the existing order information, and
extracts the common representation relating to the recheck from each of the divided groups.

5. The order creation support apparatus according to claim 3, wherein the processing circuitry
divides the existing order information corresponding to the workflow information into a plurality of groups, according to a setting content of a resource recorded in the workflow information, and
extracts the common representation relating to the resource from each of the divided groups.

6. The order creation support apparatus according to claim 5, wherein the processing circuitry
determines an operational state of the resource, based on schedule information in which a work schedule of the resource is recorded, and
displays the determined operational state of the resource, as the support information.

7. The order creation support apparatus according to claim 1, wherein the processing circuitry extracts the common representation from the existing order information, for a specific item included in the extracted existing order information.

8. The order creation support apparatus according to claim 1, wherein the processing circuitry displays the new order information and the support information on a same screen.

9. The order creation support apparatus according to claim 1, wherein the processing circuitry highlights and displays a character string corresponding to the common representation included in the new order information.

10. An order creation support method, comprising:
extracting existing order information similar to input new order information, from pieces of the existing order information, based on similarity between the new order information and the existing order information;
extracting representation commonly used for a specific item, from a character string included in the extracted existing order information, as common representation; and
displaying the new order information and support information based on the extracted common representation.
